(19) United States
(12) Patent Application Publication
(10) Pub. No.: US 2017/0156585 A1
(43) Pub. Date: Jun. 8, 2017

(54) EYE CONDITION DETERMINATION SYSTEM

(71) Applicant: KONINKLIJKE PHILIPS N.V., EINDHOVEN (NL)

(72) Inventors: WEIRAN NIE, SHANGHAI (CN); JIANFENG WANG, SHANGHAI (CN); YAO-JUNG WEN, CONCORD, CA (US); SIRISHA RANGAVAJHALA, WHITE PLAINS, NY (US); MAULIN DAHYABHAI PATEL, TUCKAHOE, NY (US); PARIKSHIT SHAH, WHITE PLAINS, NY (US)

(21) Appl. No.: 15/321,234

(22) PCT Filed: Jun. 24, 2015
(86) PCT No.: PCT/IB2015/054723

§ 371 Date: Dec. 22, 2016

(30) Foreign Application Priority Data
Jul. 2, 2014 (CN) PCT/CN2014/081519

(51) Int. Cl. A61B 3/028; A61B 3/032; A61B 3/00; A61B 3/08

(52) U.S. Cl. CPC A61B 3/028; A61B 3/085; A61B 3/032; A61B 3/0025; A61B 3/0041; A61B 3/0033

(57) ABSTRACT

The invention relates to an eye condition determination system (1) for determining an eye condition of a person, especially of a child, during a viewing activity during which the person views an object (3). A spatial characteristics capturing unit (2) captures a spatial dimension of the object and/or a spatial position of the object relative to the person during the viewing activity, and an eye condition determination unit (4) determines an eye condition like myopia or strabismus based on the captured spatial dimension of the object and/or the captured spatial position of the object relative to the person. This allows for a determination of the eye condition during a normal viewing activity like reading a text without any assistance by, for instance, parents, especially without any user interaction.

### FIELD OF THE INVENTION

The invention relates to an eye condition determination system, method and computer program for determining an eye condition of a person.

### BACKGROUND OF THE INVENTION

US 2010/0253913 A1 discloses a system for measuring the reading acuity of a person. The system shows characters having different sizes to a person and the person has to input into the system whether he can read the respective character. The system then determines the reading acuity depending on the input provided by the person and depending on the respective character size.

This system has the drawback that the measurement of the reading acuity requires a user interaction such that young children may not be able to use the system without the assistance of their parents.

### SUMMARY OF THE INVENTION

It is an object of the present invention to provide an eye condition determination system, method and computer program for determining an eye condition of a person, which allows for the determination of an eye condition of a young child without requiring the assistance of, for instance, the parents.

In a first aspect of the present invention an eye condition determination system for determining an eye condition of a person is presented, wherein the system is adapted to determine the eye condition during a viewing activity during which the person views an object and wherein the system comprises:
a spatial characteristics capturing unit for capturing a spatial dimension of the object and/or a spatial position of the object relative to the person during the viewing activity, and
an eye condition determination unit for determining the eye condition based on the captured spatial dimension of the object and/or the captured spatial position of the object relative to the person.

Since the system is adapted to determine the eye condition during a viewing activity during which the person views an object, wherein the viewing activity may be regarded as being a normal viewing activity like looking in a book, viewing a screen of a television, viewing a display of a computer, et cetera, i.e. not a specific viewing activity for determining the eye condition, wherein the spatial dimension of the viewed object, which might be a display or a book, and/or the spatial position of the viewed object relative to the person is captured during the viewing activity and wherein the eye condition like a visual acuity or strabismus is determined based on the captured visual spatial dimension of the object and/or the captured spatial position of the object relative to the person, the eye condition can be determined without any assistance by, for instance, parents, especially without any user interaction, during a normal activity.

The object to be viewed may be, for instance, a book, a blackboard, a notice board, a road sign, a paper document, a computer with a screen like a touch pad or a personal computer monitor, a television, et cetera. The visual spatial dimension of the object is preferentially a spatial dimension of a content shown on the object. For instance, if the object is a book or a computer, the visual spatial dimension can be a dimension of the content of the book or of the screen of the computer, respectively. In an embodiment the visual spatial dimension is a text font size and/or a line spacing.

In an embodiment the system comprises an image providing unit for providing an object image being an image of the object, wherein the object image has been generated by a person camera attached to the person, and/or a facial image being an image of the face of the person, wherein the facial image has been generated by an object camera attached to the object, wherein the spatial characteristics capturing unit is adapted to determine the spatial position of the object relative to the person based on the provided object image and/or the provided facial image.

The person camera is preferentially integrated with eyeglasses to be worn by the person, in order to attach the person camera to the person. For instance, the person camera may be embedded on the eyeglasses. The person camera points away from the person, if the person views the object through the eyeglasses, wherein in this embodiment the determined spatial position of the object relative to the person is preferentially the spatial position of the object relative to the eyeglasses. The object camera may be integrated with the object. For instance, the object might be a computer with a screen like a touch pad or a personal computer monitor, a television, et cetera, wherein the object camera can be a camera facing the person and built in the object.

The spatial position defines a spatial distance between the person and the object, which is preferentially used for determining the eye condition. For determining the spatial position of the object relative to the person and hence the spatial distance between the person and the object known image-based distance estimation algorithms may be used like the algorithm disclosed in EP 2 573 696 A2, which is herewith incorporated by reference.

In an embodiment the system comprises an image providing unit for providing an object image being an image of the object, wherein the spatial characteristics capturing unit is adapted to determine the spatial dimension of the object based on the provided object image. In particular, the image providing unit is adapted to provide an object image which has been generated by a person camera attached to the person. Also in this embodiment the person camera is preferentially integrated with eyeglasses to be worn by the person, in order to attach the person camera to the person. For instance, the person camera may be embedded on the eyeglasses, wherein the person camera points away from the person, if the person views the object through the eyeglasses. The object image used for determining the spatial dimension of the object may also be used for determining the position of the object relative to the person, especially the distance between the object and the person.

In a preferred embodiment the object is a computer with a display, wherein the system comprise a screenshot providing unit for providing a screenshot of the display, wherein the spatial characteristics capturing unit is adapted to determine the spatial dimension of the object based on the provided screenshot. Alternatively or in addition, the spatial characteristics capturing unit may be adapted to receive the spatial dimension via an application user interface (API) of an operating system of the computer.

In an embodiment the system comprises an image providing unit for providing a facial image being an image of the face of the person and an eyes shape determination unit for determining the shape and optionally also the size of the eyes based on the provided facial image, wherein the eye condition determination unit is adapted to determine the eye condition further based on the determined shape of the eyes and optionally also based on the determined size of the eyes. Moreover, in an embodiment the system comprises a visual axes determination unit for determining the visual axes of the eyes based on the provided facial image, wherein the eye condition determination unit is adapted to determine the eye condition further based on the determined visual axes of the eyes. By further using these parameters for determining the eye condition the eye condition may be determined more accurately and/or more different kinds of eye conditions may be determined.

The spatial characteristics capturing unit is preferentially adapted to capture an element distance between elements of the object as the visual spatial dimension of the object. In particular, the object may show characters as elements, wherein the element distance may be a distance between characters and/or a distance between words formed by the characters and/or a distance between lines formed by the characters.

The eye condition determination unit is preferentially adapted to determine the visual acuity and/or strabismus as the eye condition. For instance, the spatial characteristics capturing unit can be adapted to capture an element distance between elements of the object as the visual spatial dimension of the object and the spatial distance between the person and the object as the spatial position of the object, wherein the eye condition determination unit can be adapted to determine the visual acuity based on the element distance and the spatial distance between the person and the object. Moreover, the eye condition determination unit can be adapted to determine the visual acuity based on a) the determined shape and optionally also the determined size of the person's eye lids and b) the captured visual spatial dimension of the object and/or the captured spatial position of the object relative to the person. Furthermore, the eye condition determination unit can be adapted to determine strabismus based on whether visual axes of the eyes of the person converge at the captured spatial position of the object.

The system may further comprise a warning message generation unit for generating a warning message depending on the determined eye condition. The warning message generation unit is preferentially adapted to provide the warning message to certain persons like parents of children, of which the eye condition has been determined. In particular, the warning message generation unit can be adapted to, for instance, send the warning message as an email or in another format to the parents, if an abnormal eye condition, i.e. an eye condition deviating from a predefined eye condition, has been determined.

In a further aspect of the present invention an eye condition determination method for determining an eye condition of a person is presented, wherein the method is adapted to determine the eye condition during a viewing activity during which the person views an object and wherein the method comprises:
capturing a spatial dimension of the object and/or a spatial position of the object relative to the person during the viewing activity by a spatial characteristics capturing unit, and
determining the eye condition based on the captured visual spatial dimension of the object and/or the captured spatial position of the object relative to the person by an eye condition determination unit.

In a further aspect of the present invention a computer program for determining an eye condition of a person is presented, wherein the computer program comprises program code means for causing an eye condition determination system as defined in claim 1 to carry out the steps of the eye condition determination method as defined in claim 13, when the computer program is run on a computer controlling the eye condition determination system.

It shall be understood that the eye condition determination apparatus of claim 1, the eye condition determination method of claim 13 and the computer program of claim 14 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 schematically and exemplarily shows an embodiment of an eye condition determination system for determining an eye condition of a person,

FIGS. 2 and 3 schematically and exemplarily illustrate spatial parameters used by the eye condition determination system for determining the eye condition,

FIGS. 4 and 5 schematically and exemplarily show the shape and size of a user's eye while focusing on an object, wherein FIG. 4 shows an eye of a myopia patient and FIG. 5 shows an eye of a healthy person,

FIG. 6 schematically and exemplarily shows a further embodiment of an eye condition determination system for determining an eye condition of a person,

FIGS. 7 to 9 schematically and exemplarily illustrate different locations of an intersection of two visual axes of a person relative to a location of a target object, and

FIG. 10 shows a flowchart exemplarily illustrating an embodiment of an eye condition determination method for determining an eye condition of a person.

### DETAILED DESCRIPTION OF EMBODIMENTS

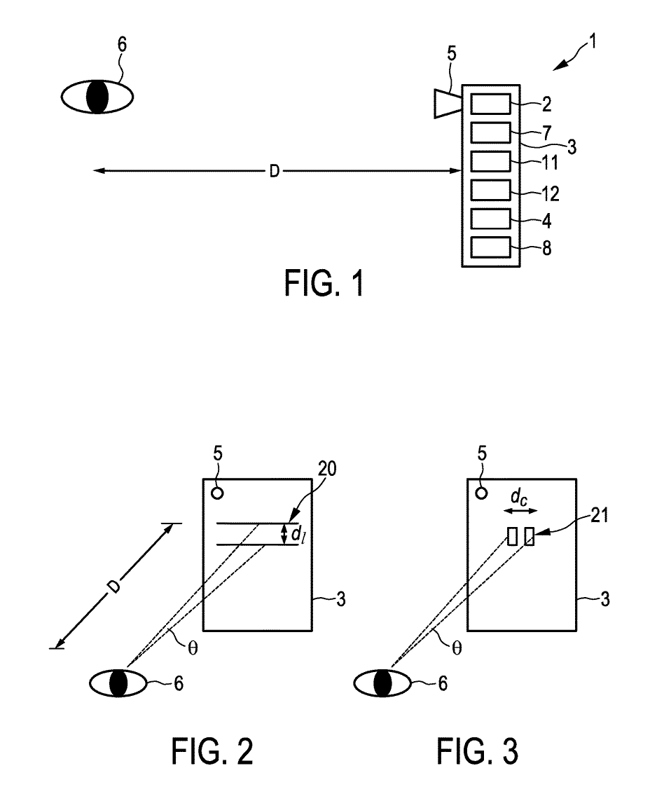

FIG. 1 shows schematically and exemplarily an embodiment of an eye condition determination system for determining an eye condition of a person. The system 1 is adapted to determine the eye condition during a viewing activity during which the person views an object 3 not for determining the eye condition, i.e. the system 1 is adapted to determine the eye condition during a normal activity of the person without distracting the person from the normal activity and without requiring any conscious actions from the person for performing an eye condition test.

The system 1 comprises a spatial characteristics capturing unit 2 for capturing a spatial dimension of the object 3 during the viewing activity. In this embodiment the object 3 is a computer with a screen like a touch pad or a personal computer monitor and the spatial characteristics capturing unit is adapted to capture a spatial dimension of a content shown by the computer 3 as the spatial dimension of the object. For instance, the spatial characteristics capturing unit 2 can be adapted to capture a text font size and/or a line spacing of a text shown by the computer 3 as the spatial dimension of the object. In this embodiment the spatial characteristics capturing unit 2 uses a screenshot of the display of the computer, which is provided by a screenshot providing unit 7, in order to determine the spatial dimension. Thus, the spatial characteristics capturing unit 2, which may also be regarded as being a viewing content capture unit, and the screenshot providing unit 7 can be software programs residing in the computer 3, wherein the software programs constantly take the screenshot of the display of the computer 3 and analyze the characteristics of the contents being displayed, in order to capture the spatial dimension, especially the text font size and/or the line spacing of a text shown on the display. In another embodiment the spatial characteristics capturing unit can also be adapted to receive the spatial dimension via an application user interface (API) of the operating system of the computer 3. Thus, for instance, the spatial characteristics capturing unit can issue function calls to learn about characteristics of the contents being displayed by using the API of the underlying operating system of the computer 3.

The spatial characteristics capturing unit 2 is further adapted to capture a spatial position of the computer 3, especially of the display of the computer 3, relative to the person during the viewing activity. In particular, the spatial characteristics capturing unit 2 is adapted to determine the spatial distance D between the display of the computer 3 and the eyes 6 of the person by using a built-in camera 5 facing the person. The built-in camera 5 can be regarded as being an image providing unit and as being an object camera that is attached to the object 3, which is adapted to provide a facial image of the person. For determining the spatial distance between the display of the computer 3 and the eyes 6 of the person based on the facial image generated by the built-in camera 5 known image-based distance estimation algorithms can be used like the algorithm disclosed in EP 2 573 696 A2.

The system 1 further comprises an eye condition determination unit 4 for determining the eye condition based on the captured spatial dimension of the object 3 and the captured spatial position of the object 3 relative to the person. In particular, the eye condition determination unit 4 is adapted to determine the visual acuity of the eyes 6 of the person based on the spatial dimension of the contents shown on the display of the computer 3 and based on the spatial distance between the eyes 6 and the display of the computer 3.

FIG. 2 schematically and exemplarily illustrates the computer 3 with the built-in camera 5, wherein on the display of the computer 3 text lines 20 are shown. The distance dₜ between the text lines 20 is captured by the spatial characteristics capturing unit 2 and used together with the spatial distance D between the eyes 6 and the display of the computer 3 for determining the visual acuity.

In order to get the best visual acuity when reading a textual document on the computer 3, the person may subconsciously adjust his/her viewing distance, or purposefully adjust, for instance, the line spacing and/or the text font size and/or the distance between adjacent characters or words, if the viewing content is electronically adjustable. The preferred distance, at which the person is reading, is the spatial distance D between the eyes 6 of the person and the display of the computer 3 calculated by the spatial characteristics capturing unit 2 by using the facial image generated by the built-in camera 5. The line spacing dₜ or, for instance, the distance d_{c} between two adjacent characters, which is schematically and exemplarily illustrated in FIG. 3, can be captured by the spatial characteristics capturing unit 2 which may be co-located with the content being displayed on the computer 3. Based on the line spacing dₜ or the distance between two adjacent characters d_{c} and the spatial distance D between the eyes 6 of the person and the display of the computer 3 the resolution angle of the person, denoted as θ (≈dₜ/D rad or ≈d_{c}/D rad), can be calculated by the eye condition determination unit 4. By monitoring the resolution angle θ over a relatively long period of time statistics regarding the resolution angle can be obtained like a maximum resolution angle, a minimum resolution angle and a mean resolution angle. The determined current resolution angle, the maximum resolution angle, the minimum resolution angle and/or the mean resolution angle can be regarded as being the eye condition determined by the eye condition determination unit. For instance, if the respective resolution value is relatively small, it can be assumed that the eyes 6 of the person have a relatively high visual acuity, whereas, if the resolution angle is relatively large, it can be assumed that the visual acuity of the eyes 6 of the person is relatively small. Thus, a person's near or far vision can be estimated, as if he/she is testing with a Snellen chart.

The system 1 further comprises an eyes shape determination unit 11 for determining the shape of the eyes 6 based on the facial image generated by the built-in camera 5. In this embodiment the eyes shape determination unit 11 is further adapted to determine the size of the eyes 6, wherein the shape and size of the eyes may be determined by determining the contour or outline of the eyes and wherein the eye condition termination unit 4 is adapted to determine the eye condition further based on the determined size and shape of the eyes 6, especially based on a determined contour or outline of the eyes.

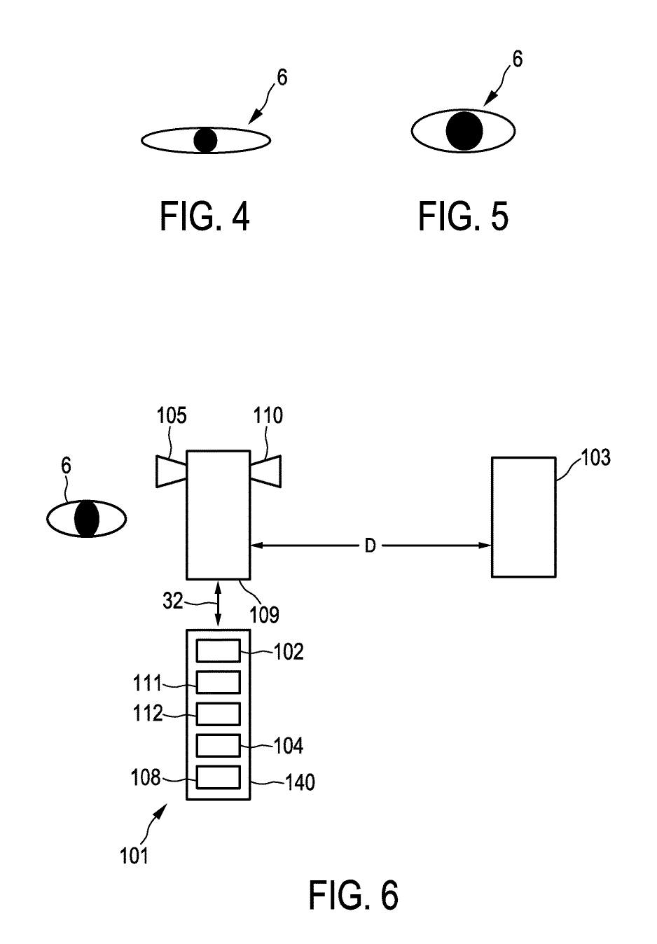

A myopia patient with no or inadequate eye glasses tends to view an object by narrowing his/her eyes so that the eyes can better focus as schematically and exemplarily illustrated in FIG. 4. If the person does not have myopia, the eyes are less narrowed during, for instance, reading a textual document as schematically and exemplarily illustrated in FIG. 5. The eyes shape determination unit 11 can therefore be adapted to detect, for instance, an eye shape change by applying an outline extraction algorithm to the facial image. If the eye narrowing event is frequently found to be associated with the event of viewing small or far away elements as detected by the spatial characteristics capturing unit 2, the person may have myopia, especially the power of the eye glasses may be inadequate. The eye condition determination unit 4 can be adapted to use a machine learning model like a Bayesian network for determining the visual acuity, wherein the machine learning model may be trained by a training data set comprising a) known distances and/or character sizes and/or line spacings and/or shapes of the eyes and/or sizes of eyes, especially contours or outlines of the eyes, and optional further parameters like the age of the person and b) known visual acuities.

The system further comprises a visual axes determination unit 12 for determining the visual axes of the eyes 6 based on the provided facial image, wherein the eye condition determination unit 4 is adapted to determine strabismus based on the determined visual axes of the eyes 6 and the spatial position of the object 3 relative to the person. In particular, the eye condition determination unit 4 can be adapted to determine whether the visual axes of the eyes 6 converge at the spatial location of the objet 3, wherein, if this is not the case, the eye condition determination unit 4 can determine that the person has strabismus.

The system 1 further comprises a warning message generation unit 8 for generating a warning message depending on the determined eye condition. For instance, the warning message generation unit 8 can be adapted to generate a warning message, if myopia and/or strabismus has been determined by the eye condition determination unit 4. The warning message generation unit 8 can be adapted to send warning messages to specific recipients like parents, if the eye condition determination unit 4 has detected that a child has myopia and/or strabismus. The warning message can be, for instance, an acoustical warning message and/or an optical warning message. The warning message can also be a textual message which is sent to, for example, an email address of the parents.

FIG. 6 schematically and exemplarily illustrates a further embodiment of an eye condition determination system for determining an eye condition of a person. Also in this embodiment the system 101 is adapted to determine the eye condition during a normal viewing activity during which the person views an object not for determining the eye condition, i.e. not during performing an eye condition test, but during, for instance, reading a textual document. The eye condition determination system 101 comprises a spatial characteristics capturing unit 102 for capturing a spatial dimension of an object 103 and for capturing the spatial position of the object 103 relative to the person during the viewing activity. More specifically, in this embodiment eyeglasses 109 are worn by the person, wherein a person camera 110 is attached to the eyeglasses 109. The person camera 110 points outward, i.e. towards the object 103 being, in this embodiment, an object having a textual content. For instance, in this embodiment the object 103 might be a book, a blackboard, a notice board, a paper document, a road sign, et cetera. The person camera 110 is used for generating an object image, wherein the spatial characteristics capturing unit 102 is adapted to determine the spatial dimension of the object, especially of the contents shown by the object like character sizes, lines spacings et cetera, based on the object image. The spatial characteristics capturing unit 102 is further adapted to determine the spatial distance between the person and the object 103, i.e. in this embodiment the spatial distance D between the eyeglasses 109 worn by the person and the object 103, based on the object image by using known image-based distance estimation techniques. The system 101 further comprises an eye condition determination unit 104 which can be adapted to determine, for instance, the visual acuity based on the spatial distance D between the eyeglasses 109 and the object 103 and a line spacing and/or a distance between two adjacent characters as described above with reference to FIGS. 2 and 3.

The eyeglasses 109 comprise further cameras 105 for generating facial images of the person, wherein the facial images show at least the part of the face comprising the eyes 6. In particular, the eyeglasses 109 may comprise two further cameras 105, wherein each camera 105 generates a facial image showing an eye of the person, i.e. in this embodiment the facial images do not need to be images showing the entire face of the person, but they can be images showing at least the parts of the face which comprise the eyes 6.

The facial images generated by the cameras 105 can be used by an eyes shape determination unit 111 for determining the size and shape of the eyes 6, wherein this size and shape of the eyes 6 can be used by the eye condition determination unit 104 for determining an eye condition like myopia as described above with reference to FIGS. 4 and 5. Moreover, a visual axes determination unit 112 can determine the visual axes of the eyes 6 based on the provided facial images, wherein the eye condition determination unit 104 can be adapted to determine strabismus by determining whether the visual axes of the eyes 6 converge at the spatial position of the object 103 provided by the spatial characteristics capturing unit 102. For instance, the facial images may two-dimensional or three-dimensional images, wherein the visual axes determination unit 112 can be adapted to determine the position of the respective pupil relative to the location of the outline of the respective eye, i.e. relative to the eye lid, in the respective facial image and to determine the respective visual axis based on this relative position. If the facial image is a three-dimensional image, the visual axes determination unit 112 may be adapted to determine the protrusion of the respective eyeball, especially by using a depth extraction technique, in order to determine the normal axis of the respective eyeball, which can be used as the respective visual axis.

The spatial characteristics capturing unit 102 is preferentially adapted to provide the spatial position of the object based on an image of the object. If the spatial characteristics capturing unit 102 identifies several objects in the image, it may use a motion tracking algorithm for identifying the object in focus. In particular, the spatial characteristics capturing unit 102 can be adapted to detect the motions of the objects identified in the image, to detect the motion of the eyeballs of the person based on the facial images and to try to correlate or match the motion of the eyeballs with each of the motions of the different objects. The object, for which the best motion correlation or motion matching could be achieved, can be determined as being the object in focus, wherein the two visual axes should intersect at the position of the object in focus.

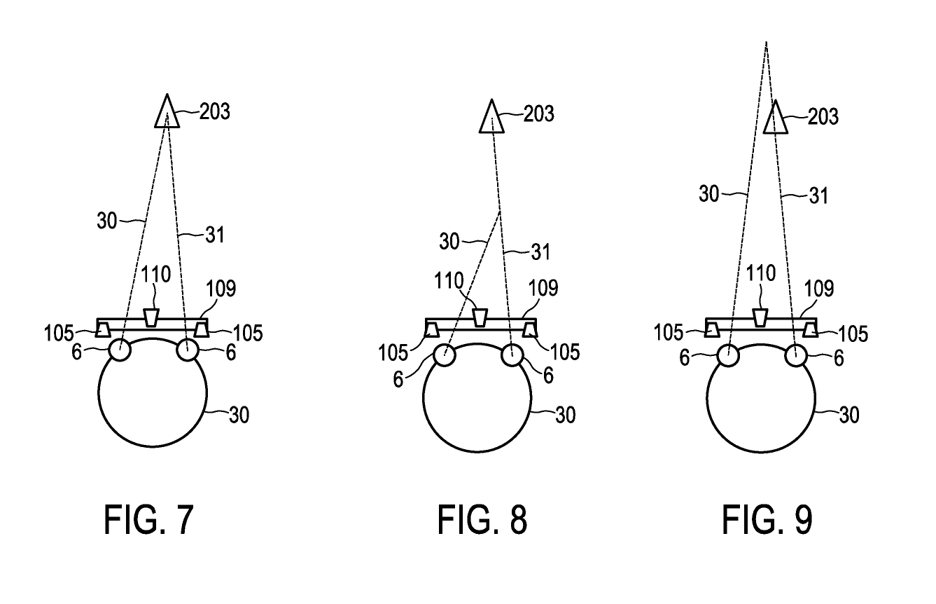

FIGS. 7 to 9 exemplarily illustrate how the symptoms of strabismus can be determined by the eye condition determination system. FIG. 7 illustrates the situation how normal, i.e. healthy, eyes 106 of a person 30 view a target object 203. The visual axes 30, 31 intersect at the target object 203 being focused. FIG. 8 shows the so-called “cross-eyed” strabismus symptom and FIG. 9 shows the so-called “wall-eyed” strabismus symptom. By determining the location of the intersection of the two visual axes 30, 31 with respect to the location of the target object 203 the eye condition determination unit 104 can distinguish between the different symptoms of strabismus, i.e. the eye condition determination unit 104 can detect the misalignment of the two visual axes 30, 31 and detect strabismus based on this misalignment.

The system 101 further comprises a warning message generation unit 108 being similar to the warning message generation unit 8 described above with reference to FIG. 1. The spatial characteristics capturing unit 102, the eyes shape determination unit 111, the visual axes determination unit 112, the eye condition determination unit 104 and the warning message generation unit 108 can be implemented in a computer 140, which communicates via a wired or wireless data connection 32 with the cameras 105, 110 of the eyeglasses 109, in order to receive the respective images which are used by the different units of the computer 140 for determining, for instance, a spatial distance between the eyeglasses 109 and the object 103, a line spacing, a distance between adjacent characters, visual axes of the eyes, the size and shape of the eyes, et cetera.

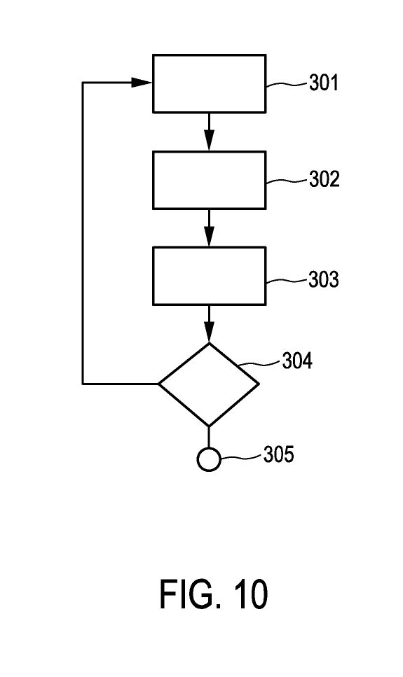

In the following an embodiment of an eye condition determination method for determining an eye condition of a person will exemplarily be described with reference to a flowchart shown in FIG. 10.

The eye condition determination method is adapted to determine the eye condition during a viewing activity during which the person views an object not for determining the eye condition, i.e. the method is adapted to determine the eye condition during a normal viewing activity of the person like reading a textual document. In step 301 the spatial dimension of the object and/or the spatial position of the object relative to the person during the viewing activity is captured by a spatial characteristics capturing unit. For instance, by using the system 1 described above with reference to FIG. 1 or the system 101 described above with reference to FIG. 6 a spatial distance between the object and the person can be determined and a line spacing or a distance between two adjacent characters of a textual document can be determined. Optionally, in step 301 further characteristics can be determined like the size and shape of the eyes.

In step 302 the eye condition is determined based on the captured visual spatial dimension of the object and/or the captured spatial position of the object relative to the person by an eye condition determination unit. For instance, the visual acuity of the person can be determined based on a captured spatial distance between the object and the person and a captured line spacing or a captured distance between two adjacent characters of a textual document shown by the object. In step 302 also other eye conditions can be determined like strabismus. Moreover, additional characteristics can be used for determining the eye condition like the size and shape of the eyes of the person.

In step 303 it is determined whether the eye condition is abnormal such that a warning message needs to be generated by a warning message generation unit. For instance, if in step 302 the visual acuity is determined by determining a resolution angle, a warning message can be generated, if the resolution angle is not within a predefined resolution angle range which correspond to a normal eye condition. Moreover, if in step 302 strabismus has been investigated, a warning message may be generated, if the visual axes of the eyes do not intersect at the location of the object.

In step 304 it is determined whether an abort criterion has been fulfilled. The abort criterion may be, for instance, whether a user has input a stop command into the system. If the abort criterion is fulfilled, the method ends in step 305. Otherwise, the method continues with step 301. Thus, the eye condition may be determined continuously over a large period of time, for instance, over some days, months or even years, until it is indicated that the eye condition determination method should stop.

Myopia, or near-sightedness, is a problematic eye condition where incoming light does not focus on the retina but in front of it. Another undesirable eye condition is strabismus, which is a condition in which two eyes are not properly aligned with each other when looking at an object. Young children are especially prone to these eye conditions during their visual development. Improper reading posture, lack of exercise, overburdening school work, et cetera can drastically increase the chance of developing myopia. Early detection of problematic eye symptoms is the key to carrying out correct treatment and preventing the eyes from further deteriorating. However, complete eye examination is usually done only once or twice a year for a child in an average family and is only available at a hospital or optometrists via dedicated examination equipment. A naked eye examination may be carried out by parents. But, such a naked eye examination cannot always detect early signs of a child's eye condition, because some symptoms only manifest themselves when the child is doing a certain kind of activity like focusing on an object. Furthermore, some parents are simply too busy to attend their children or lack certain eye healthcare knowledge. The eye condition determination systems described above with reference to FIGS. 1 and 6 are therefore adapted to detect early signs of eye diseases and to give warnings to the parents. The system can be built as an add-on to a computer like a tablet computer, a smart phone or a personal computer, or to a television, to eyeglasses et cetera. By monitoring the children's everyday vision-related activities such as reading, doing homework, playing video games et cetera early signs of eye conditions can be detected and their parents can be warned. If there is such a warning, more thorough and professional examinations can be carried out for further evaluation. As a result, problematic eye conditions of persons, especially of young children, can be diagnosed more timely.

The spatial characteristics capturing unit can be adapted to capture static and/or dynamic spatial characteristics. For instance, the spatial characteristics capturing unit can be adapted to capture a changing distance between the person and the object and/or a changing line spacing or distance between adjacent characters of a textual document shown by the object.

The systems are preferentially adapted to determine the eye condition based on image processing. The described eye condition determination technique may be applied to in-home children eyesight monitoring, children eye care and smart homes in general. It preferentially detects a child's vision problem without interfering with his/her normal everyday activity.

Although in above described embodiments the eye condition determination unit is adapted to determine certain eye conditions like the visual acuity and strabismus, in other embodiments the eye condition determination unit can be adapted to determine other eye conditions. Moreover, although in above described embodiments certain objects have been viewed by the person while determining the eye condition, in other embodiments also other objects can be viewed while determining the eye condition. For instance, instead of a computer with a display a television can be viewed by the person while determining the eye condition.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the capturing of a spatial dimension of an object, i.e. for instance of a line spacing, a distance between adjacent characters, a distance between adjacent words, the determination of the eye condition, the determination of the size and shape of the eyes, the determination of the visual axes of the eyes, et cetera performed by one or several units or devices can be performed by any other number of units or devices. These procedures and/or the control of the eye condition determination system in accordance with the eye condition determination method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to an eye condition determination system for determining an eye condition of a person, especially of a child, during a viewing activity during which the person views an object. A spatial characteristics capturing unit captures a spatial dimension of the object and/or a spatial position of the object relative to the person during the viewing activity, and an eye condition determination unit determines an eye condition like myopia or strabismus based on the captured spatial dimension of the object and/or the captured spatial position of the object relative to the person. This allows for a determination of the eye condition during a normal viewing activity like reading a text without any assistance by, for instance, parents, especially without any user interaction.

1. An eye condition determination system for determining an eye condition of a person, wherein the system is adapted to determine the eye condition during an everyday viewing activity during which the person views an object and wherein the system comprises:
a spatial characteristics capturing unit for capturing a spatial dimension of the object viewed by the person and/or a spatial position of the object relative to the person during the everyday viewing activity, and
an eye condition determination unit for determining the eye condition based on the spatial dimension of the object captured during the everyday viewing activity and/or the spatial position of the object relative to the person captured during the everyday viewing activity.

2. The eye condition determination system as defined in claim 1, wherein the system further comprises an image providing unit for providing an object image being an image of the object, wherein the object image has been generated by a person camera attached to the person, and/or a facial image being an image of the face of the person, wherein the facial image has been generated by an object camera attached to the object, wherein the spatial characteristics capturing unit is adapted to determine the spatial position of the object relative to the person based on the provided object image and/or the provided facial image.

3. The eye condition determination system as defined in claim 1, wherein the system further comprises an image providing unit for providing an object image being an image of the object, wherein the spatial characteristics capturing unit is adapted to determine the spatial dimension of the object based on the provided object image.

4. The eye condition determination system as defined in claim 3, wherein the image providing unit is adapted to provide an object image which has been generated by a person camera attached to the person.

5. The eye condition determination system as defined in claim 1, wherein the object is a computer with a display, wherein the system comprise a screenshot providing unit for providing a screenshot of the display, wherein the spatial characteristics capturing unite is adapted to determine the spatial dimension of the object based on the provided screenshot.

6. The eye condition determination system as defined in claim 1, wherein the object is a computer with a display, wherein the spatial characteristics capturing unit is adapted to receive the spatial dimension via an application user interface (API) of an operating system of the computer.

7. The eye condition determination system as defined in claim 1, wherein the system further comprises an image providing unit for providing a facial image being an image of the face of the person and an eyes shape determination unit for determining the shape of the eyes based on the provided facial image, wherein the eye condition determination unit is adapted to determine the eye condition further based on the determined shape of the eyes.

8. The eye condition determination system as defined in claim 1, wherein the system further comprises an image providing unit for providing a facial image being an image of the face of the person and a visual axes determination unit for determining the visual axes of the eyes based on the provided facial image, wherein the eye condition determination unit is adapted to determine the eye condition further based on the determined visual axes of the eyes.

9. The eye condition determination system as defined in claim 1, wherein the spatial characteristics capturing unit is adapted to capture an element distance between elements of the object as the visual spatial dimension of the object

10. The eye condition determination system as defined in claim 9, wherein the object shows characters as elements and wherein the element distance is a distance between characters and/or a distance between words formed by the characters and/or a distance between lines formed by the characters.

11. The eye condition determination system as defined in claim 1, wherein the eye condition determination unit is adapted to determine the visual acuity and/or strabismus as the eye condition.

12. The eye condition determination system as defined in claim 1, wherein the system further comprises a warning message generation unit for generating a warning message depending on the determined eye condition.

13. An eye condition determination method for determining an eye condition of a person, wherein the method is adapted to determine the eye condition during a everyday viewing activity during which the person views an object and wherein the method comprises:
capturing a spatial dimension of the object viewed by the person and/or a spatial position of the object relative to the person during the everyday viewing activity by a spatial characteristics capturing unit, and
determining the eye condition based on the visual spatial dimension of the object captured during the everyday viewing activity and/or the spatial position of the object relative to the person by an eye condition determination unit captured during the everyday viewing activity.

14. A computer program for determining an eye condition of a person, the computer program comprising program code means for causing an eye condition determination system as defined in claim 1 to carry out the steps of the eye condition determination method, when the computer program is run on a computer controlling the eye condition determination system.
